(19) United States
(12) Patent Application Publication
(10) Pub. No.: US 2026/0290152 A1
(43) Pub. Date: Sep. 24, 2026

(54) RADIATION DETECTOR, SYSTEM, AND IMAGING METHOD

(71) Applicant: VAREX IMAGING CORPORATION, Salt Lake City, UT (US)

(72) Inventors: Marcelo C. Costa, Draper, UT (US); Andrew Hartmann, Midvale, UT (US)

(73) Assignee: VAREX IMAGING CORPORATION, Salt Lake City, UT (US)

(21) Appl. No.: 19/084,252

(22) Filed: Mar. 19, 2025

(51) Int. Cl. G08C 19/00; G01R 31/66

(52) U.S. Cl. CPC G08C 19/00; G01R 31/66

(57) ABSTRACT

Radiation imaging detectors, systems, and methods for radiation imaging are disclosed. A non-transitory computer-readable medium storing code for radiation imaging is disclosed. The code can include instructions executable by a processor to detect a connection status of an electrical connection between a radiation detector and a dock, enter an autonomous trigger mode in response to detection of the electrical connection being disconnected, and automatically acquire images through the radiation detector while the radiation detector is in the autonomous trigger mode.

### FIELD

The described embodiments relate generally to radiation imaging detectors, systems, and methods for radiation imaging. More particularly, the described embodiments relate to radiation imaging detectors that can perform radiation imaging while not connected to other components of a system, and systems and methods that perform the radiation imaging.

### BACKGROUND

Radiation detectors can be used to generate two-dimensional images or video in response to incident radiation. Radiation detectors can be used in a variety of contexts, including medical and industrial imaging. In some applications, radiation detectors can be moved between one or more fixed locations and innumerable flexible locations between the fixed locations. The radiation detectors can be used to acquire radiation images in both the fixed locations and the flexible locations. However, wired, wireless, or any other type of communication protocols that enable the communication and transfer of data and images from the radiation detectors to displays and other systems often result in the addition of multiple components, cost, and complexity to portable radiation detectors.

### SUMMARY

An aspect of the present disclosure relates to a non-transitory computer-readable medium storing code for radiation imaging. The code can include instructions executable by a processor to detect a connection status of an electrical connection between a radiation detector and a dock. An autonomous trigger mode can be entered in response to detection of the electrical connection being disconnected. Images can be automatically acquired through the radiation detector while the radiation detector is in the autonomous trigger mode.

In one or all examples, the code can be further configured to cause the radiation detector to begin an image acquisition upon detecting incident radiation and can stop the image acquisition upon detecting cessation of the incident radiation. In one or all examples, the code can further include instructions executable by the processor to store the images on a memory in the radiation detector.

In one or all examples, the code can further include instructions executable by the processor to download the images acquired by the radiation detector through the dock in response to detection of the electrical connection being connected. In one or all examples, the code can further include instructions executable by the processor to transition from the autonomous trigger mode to a connected mode in response to detection of the electrical connection being connected.

In one or all examples, the images can be acquired by an imaging array of the radiation detector. The code can further include instructions executable by the processor to power the imaging array by a supercapacitor of the radiation detector.

Another aspect of the present disclosure relates to a method of radiation imaging. The method can include detecting a status of an electrical connection between a radiation detector and a dock. Images can be transferred from the radiation detector to a computing device through the electrical connection upon detecting the radiation detector has been electrically connected to the dock.

In one or all examples, the method can further include automatically transitioning an image acquisition mode of the radiation detector upon detecting the radiation detector has been electrically disconnected from the dock. In one or all examples, when the radiation detector is electrically disconnected from the dock, the radiation detector can be configured to automatically begin an image acquisition upon detecting incident radiation and stop the image acquisition upon detecting cessation of the incident radiation.

In one or all examples, the method can further include identifying the dock with the radiation detector; and determining a location of the detector based on the identification. In one or all examples, the method can further include requesting a user action upon detecting the radiation detector has been electrically connected to the dock. In one or all examples, the method can further include assigning one or more subject identities to the images transferred through the electrical connection.

Yet another aspect of the present disclosure relates to a radiation detector. The radiation detector can include a housing, an imaging array, a connector, a memory, and a processor. The imaging array can be within the housing and the connector can be in the housing. The processor can be coupled to the imaging array, the connector, and the memory. The processor can be configured to adjust a mode of the radiation detector based on a connection status of the connector, acquire images from the imaging array based on the mode, and store the images to the memory.

In one or all examples, the processor is further configured to transmit the images from the memory to an external computing device based on the connection status of the connector. In one or all examples, the processor is configured to transfer the images stored in the memory through the connector. In one or all examples, the housing is free of a wireless communication device. In one or all examples, the processor is further configured to acquire the images by initiating an image acquisition upon the imaging array detecting incident radiation and stopping the image acquisition upon the imaging array detecting cessation of the incident radiation.

In one or all examples, the radiation detector can further include a supercapacitor in the housing. The supercapacitor can be configured to power the imaging array, power the processor, and receive power through the connector. In one or all examples, the radiation detector can further include a battery in the housing. The battery can be configured to power the imaging array, power the processor, and receive power through the connector. In one or all examples, the connector can include at least 26 pins.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 illustrates a perspective view a radiation imaging system.

FIG. 2 illustrates a schematic view of a connection between a radiation detector and a dock.

FIG. 3 illustrates a block diagram of a method of radiation imaging.

FIG. 4 illustrates a block diagram of a radiation detector that supports techniques for radiation imaging.

FIG. 5 illustrates a block diagram of a computing device that supports techniques for radiation imaging.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates to radiation imaging detectors, systems, and methods for radiation imaging. Radiation detectors can be used to scan or inspect internal structures of subjects (e.g., patients), objects, and the like. Radiation imaging systems and methods can be used for imaging in a variety of contexts, including, but not limited to, medical imaging, diagnostics, non-destructive testing, materials detection or analysis, security inspection, and the like. In a medical setting, radiation imaging systems and methods can be used to create images of a body's internal structures. In an industrial setting, radiation imaging systems and methods can be used to image internal structures in baggage, cars, cargo, other containers and objects, and the like.

More specifically, the following disclosure relates to radiation detectors and radiation imaging systems that perform operations automatically in response to a connection status of a radiation detector and methods for performing the same. A radiation detector can be connected to a dock, which can provide a connection between the radiation detector and external devices, such as a computing device. The dock can be positioned in a wall stand, in or on a table, in a mobile cart, or the like. When the radiation detector is connected to the dock, the radiation detector can be in a fixed or relatively fixed location and can communicate directly with a desired computing device. However, it can be desirable to operate the radiation detector with greater flexibility and in various positions that cannot be achieved while the radiation detector is connected to the dock. As such, it can be desirable to operate the radiation detector without the radiation detector being connected to the dock. One solution to allow the radiation detector to be used while not physically connected to the dock is to wirelessly connect the radiation detector to external devices, such as the computer. However, including wireless components in the radiation detector can increase the cost of the radiation detector and can add certification requirements for the radiation detector.

In the present disclosure, when the radiation detector is disconnected from the dock, the radiation detector can transition from a connected mode to an automated trigger mode (e.g., entering the automated trigger mode). In the automated trigger mode, the radiation detector can be armed such that the radiation detector can detect incident radiation. The radiation detector can begin an image acquisition process upon detecting incident radiation and can stop the image acquisition process upon cessation of the incident radiation or after a prescribed time. The radiation detector can include a memory on which images obtained while the radiation detector is disconnected from the dock can be stored. As such, the radiation detector can operate while not connected to the dock or any other external devices or components, and the radiation detector can be used in any desired position. The radiation detector can be free of, or not include, any wireless communication devices or components, which can reduce the cost of the radiation detector and can reduce certification requirements for the radiation detector. The radiation detector can be powered by a supercapacitor, which can increase the speed of operations performed within the radiation detector and can further reduce transportation, storage, and other requirements for the radiation detector. The radiation detector can include other built-in power sources, such as batteries or the like.

In one or all examples, when the radiation detector is connected to the dock, the radiation detector can transmit images stored on the memory of the radiation detector through a physical connector and the dock to an external device, such as a computer. The images can be transmitted automatically or can prompt a user of the external device to accept transmission of the images. This reduces steps taken by a user to obtain images from the radiation detector. The radiation detector can also transition from the automated trigger mode to the connected mode when a connection to the dock is detected. The radiation detector can be connected to the dock by a connector including 26 or more pins, which can allow for power to be provided to the radiation detector, allow for images to be transmitted through the connector, and allow for control or other signals to be transmitted between the dock and the radiation detector.

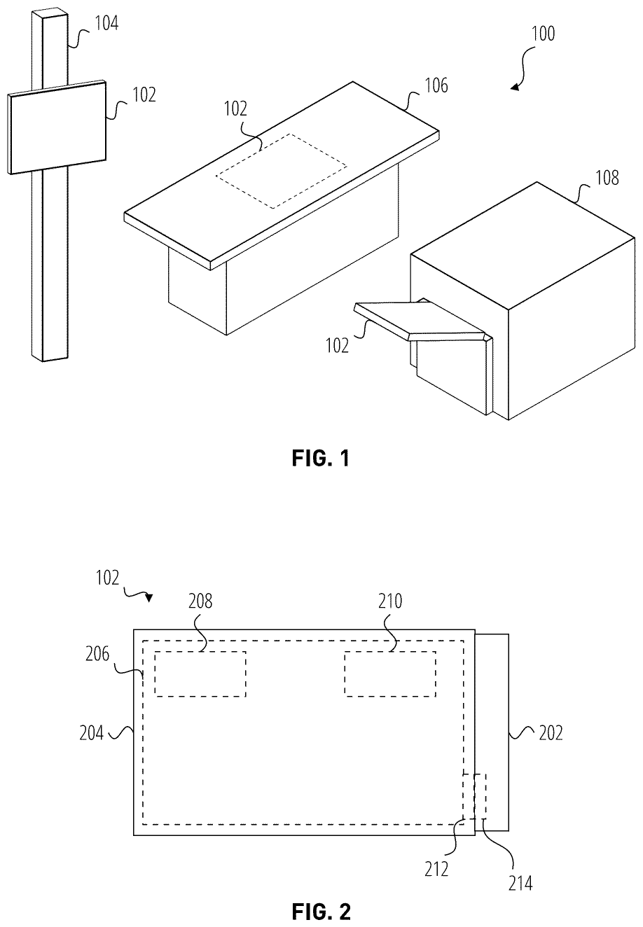

These and other examples are discussed below with reference to FIGS. 1 through 5. However, those skilled in the art will readily appreciate that the detailed description given herein with respect to these figures is for explanatory purposes only and should not be construed as limiting. Furthermore, as used herein, a system, a method, an article, a component, a feature, or a sub-feature including at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates a perspective view of an imaging system 100. The imaging system 100 can be used to detect radiation, and can be used in a variety of contexts, including medical imaging, diagnostics, radiotherapy, non-destructive testing, materials detection or analysis, security inspection, and the like. The imaging system 100 can include a radiation detector 102, which is a device configured to acquire data in response to incident radiation (e.g., x-rays or the like). In other words, the radiation detector 102 can perform radiation imaging in response to incident radiation detected by the radiation detector 102. The radiation detector 102 can generate data including image data, video data, and the like.

The radiation detector 102 can be used while connected to other components, or while the radiation detector 102 is not connected to other components. FIG. 1 illustrates that the radiation detector 102 can be connected to a wall stand 104, a table 106, or a mobile cart 108. However, the imaging system 100 of FIG. 1 is merely exemplary, and the radiation detector 102 can be connected to any suitable stands, docks, access points, or the like. When the radiation detector 102 is connected to the wall stand 104, the radiation detector 102 can be positioned vertically in the wall stand 104 (with a major surface and imaging axis of the radiation detector 102 parallel to gravity), a subject (e.g., a patient) or object can be positioned in front of the wall stand 104, and radiation imaging can be performed through the subject or object. When the radiation detector 102 is connected to the table 106, the radiation detector 102 can be positioned horizontally in the table 106 (with a major surface and imaging axis of the radiation detector 102 perpendicular to gravity), a subject or object can be positioned on the table 106, and radiation imaging can be performed through the subject or object. The mobile cart 108 can be used to charge the radiation detector 102, provide communication between the radiation detector 102 and external devices (e.g., computing devices and the like), and the like, and the radiation detector 102 can be positioned in any suitable position relative to the mobile cart 108. In one or all examples, the radiation detector 102 can determine a location of the radiation detector 102 depending on an identity of a wall stand 104, table 106, mobile cart 108, or other dock to which the radiation detector 102 is connected.

Each of the wall stand 104, the table 106, and the mobile cart 108 can include a dock to which the radiation detector 102 can be connected. Additional details of the radiation detector 102 and the dock are discussed below with respect to FIG. 2. The dock can provide a physical and electrical connection or coupling between the radiation detector 102 and external devices, such as computing devices. The radiation detector 102 can be charged through the dock, can transfer images to the external devices through the dock, and can be transitioned between modes based on a detected connection status with the dock.

For example, when the radiation detector 102 is connected with a dock, the radiation detector 102 can operate in a connected mode. In the connected mode, the radiation detector 102 can perform image acquisition in response to signals received through the dock. In one or all examples, the radiation detector 102 can perform image acquisition in response to incident radiation detected by the radiation detector 102. The radiation detector 102 in connected mode can transfer images through the dock as soon as each image acquisition is completed, or as an image is being acquired.

When the radiation detector 102 is disconnected from the dock, the radiation detector 102 can be transitioned to an automated trigger mode (e.g., enter the automated trigger mode). In some or all examples, when the radiation detector 102 is in automated trigger mode, the radiation detector 102 can be disconnected from all outside connections, including wired and wireless connections. In the automated trigger mode, the radiation detector can be armed or active and can constantly or periodically scan for incident radiation. The radiation detector 102 can start image acquisitions upon detecting incident radiation and can stop image acquisitions upon detecting cessation of incident radiation, after a prescribed time period, or the like. The radiation detector 102 can store acquired images, and any associated contextual information such as time or location, within a memory of the radiation detector 102. This allows the radiation detector 102 to perform radiation imaging in any desired position, without requiring any connection to external components or devices. The radiation detector 102 can be used to perform a desired number of image acquisitions and can then be reconnected to a dock, such as in the wall stand 104, the table 106, or the mobile cart 108.

When the radiation detector 102 is reconnected to the dock, images stored in the memory of the radiation detector 102 can be transferred to an external device through the dock. The images can be transferred automatically, or connecting the radiation detector 102 to the dock can cause the system to generate a prompt to the user via an interface, requesting instructions from the user for transferring the images. This allows images to be acquired by the radiation detector 102 and transferred to an external device, without requiring any wireless communication components within the radiation detector 102. Moreover, the radiation detector 102 can be used while disconnected from external devices, which allows the radiation detector 102 to be used in any desired position relative to a subject or object.

FIG. 2 illustrates a schematic view of a connection between a radiation detector 102 and a dock 202. The radiation detector 102 can be the same as or similar to the radiation detector 102 discussed above with respect to FIG. 1. The dock 202 can be included in each of the wall stand 104, the table 106, and the mobile cart 108, or can be a component that connects the radiation detector 102 to any external device, such as a computing device.

The radiation detector 102 can include a housing 204, an imaging array 206, a memory 208, an energy storage 210, and a connector 212. The housing 204 can define an internal volume in which the imaging array 206, the memory 208, and the energy storage 210 can be positioned and mounted. The imaging array 206 can be configured to generate an image in response to incident radiation. The imaging array 206 can include a variety of sensors configured to generate data based on incident radiation. The imaging array 206 can include any combination of direct conversion sensors, indirect conversion sensors, photon counters, radiation conversion materials (e.g., scintillator materials), or the like.

The memory 208 can store images or data acquired by the imaging array 206 as the radiation detector 102 performs image acquisitions. The memory 208 can include random access memory (RAM), read only memory (ROM), or the like. The memory 208 can be a volatile memory, a non-volatile memory, or can include both volatile and non-volatile memory. The memory 208 can further store computer-readable, computer-executable software including instructions that, when executed by a processor, cause the processor to perform various functions described herein. For example, the memory 208 can store software used to perform image acquisition through the imaging array 206, to adjust the mode of the radiation detector 102, to operate the radiation detector 102 based on the current operating mode of the radiation detector 102, to transfer images or data from the memory 208 to external components or devices, and the like. In one or all examples, the memory 208 can store a basic input/output system (BIOS), which can control interactions with peripheral components or devices. The memory 208 can be used to store images acquired by the radiation detector 102 while the radiation detector 102 is not connected to external components or devices, which facilitates the use of the radiation detector 102 in any desired position. The images stored on the memory 208 can then be transferred to external components or devices by connecting the radiation detector 102 to the dock 202.

The energy storage 210 can power the electrical components of the radiation detector 102 to operate while the radiation detector 102 is not connected to an external power source. For example, the radiation detector 102 can power the imaging array 206 and the memory 208. In some or all examples, the energy storage 210 can include a supercapacitor, which can be charged quickly when the radiation detector 102 is connected to the dock 202 and can quickly supply power to components of the radiation detector 102, such as the imaging array 206. For example, the energy storage 210 can provide power to the imaging array 206 quickly upon detection of incident radiation. The energy storage 210 can support a charge sufficient to perform a large number of image acquisitions, such as tens or hundreds of image acquisitions. In one or all examples, the radiation detector 102 can be free from batteries (e.g., the energy storage 210 can include a supercapacitor or other energy storage), which can reduce the requirements on storage, transportation, and the like of the radiation detector 102. In one or all examples, the energy storage 210 can include a battery or other built-in power or energy storage device.

The radiation detector 102 can be connected to the dock 202 through the connector 212 and a connector 214 of the dock 202. The connectors 212, 214 can be mated connectors. In one or all examples, the connector 212 can be a female connector or socket and the connector 214 can be a male connector or plug. However, the connector 212 can be a plug and the connector 214 can be a socket, or other known connectors (such as biased pin connectors or pogo pins) can be used by the present system. The connectors 212, 214 can include 26 pins or a greater number than 26 pins, and can be used to transfer power, imaging data, signals, and commands between the radiation detector 102 and the dock 202 (and external devices connected to the dock 202). In one or all examples, the radiation detector 102 can be free from wireless communication devices (e.g., Wi-Fi, Bluetooth, radio, infrared communication, or other wireless communication components). This can reduce the cost of the radiation detector 102 and reduce certification requirements for the radiation detector 102. In one or all examples, the housing 204 can be free from wireless communication devices, but the radiation detector 102 can be connected to external devices by wireless communication through the dock 202.

The radiation detector 102 and/or an external device (e.g., a computing device) connected to the dock 202 can monitor a connection status of the connection between the connector 212 and the connector 214. The radiation detector 102 can operate in a connected mode when the connector 212 is connected to the connector 214, operate in an automated trigger mode when the connector 212 is not connected to the connector 214, and transition between the connected mode and the automated trigger mode when the connector 212 and the connector 214 are connected or disconnected. Transitions between the connected mode and the automated trigger mode can be automatic in response to the radiation detector 102 detecting that the connection status between the connector 212 and the connector 214 has changed. Images stored on the radiation detector 102 can be transmitted or uploaded through the connectors 212, 214 when the connector 212 is connected to the connector 214. This transmission can be automatic, as the radiation detector 102 is connected to the dock 202. In one or all examples, connecting the connector 212 to the connector 214 can trigger prompts to a user interface connected to the dock 202, allowing a user to accept transmission of the images. In other words, a handshaking process can be performed by the radiation detector 102 and/or an external device connected to the dock 202, which can be used in the transmission of images from the memory 208 of the radiation detector 102 through the dock 202 to the external device. Further prompts, such as whether all images relate to a single subject or object, the identity of a subject or object associated with each image, and the like can also be provided to the user via an interface. Performing various functions of the radiation detector 102 automatically in response to the connection status of the connectors 212, 214 allows for the radiation detector 102 to be used while not connected to external components or devices, in any position, without requiring action by a user, to acquire a desired number of radiation images of one or more subjects or objects.

In one or all examples, the radiation detector 102 can determine a location of the radiation detector 102 depending on an identity of the dock 202 to which the radiation detector 102 is connected. For example, the dock 202 can be included in a wall stand 104, a table 106, a mobile cart 108, or can be otherwise positioned or located and the radiation detector 102 can determine the location of the radiation detector 102 based on the dock 202 to which the radiation detector 102 is connected.

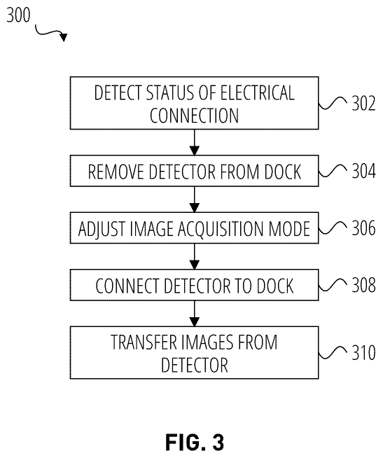

FIG. 3 illustrates a block diagram of a method 300 of radiation imaging. The method 300 can be used with the radiation detectors 102 and in the imaging system 100, discussed above with respect to FIGS. 1 and 2. The method 300 can include a block 302 in which a status of an electrical connection between a radiation detector and a dock is detected; a block 304 in which the radiation detector is removed from the dock; a block 306 in which an image acquisition mode of the radiation detector is adjusted; a block 308 in which the radiation detector is connected to the dock; and a block 310 in which images stored in the radiation detector are transferred.

In block 302, a status of an electrical connection between a radiation detector and a dock is detected. As discussed above in reference to FIG. 2, the radiation detector can be physically and electrically coupled to the dock by a connector. The connector can include 26 pins or more, and can be used to transfer power, imaging data, signals, and commands for components of the imaging system, and the like between the radiation detector and the dock. The status of the connection between the radiation detector and the dock can be constantly monitored, such as throughout the method 300. The status of the connection between the radiation detector and the dock can be monitored by the radiation detector and/or the dock, which can include by components connected to the dock. The dock can include a dock provided in any component of the imaging system, such as a wall stand, a table, a mobile cart, another bucky, or the like.

In block 304, the radiation detector is removed from the dock. Block 302 can detect that the radiation detector has been removed from the dock. In response to the radiation detector being removed from the dock in block 304, an image acquisition mode of the radiation detector is adjusted in block 306. The image acquisition mode of the radiation detector can be adjusted or transitioned to an armed or autonomous trigger mode. The autonomous trigger mode can also be referred to as an unconnected mode. The image acquisition mode of the radiation detector can be transitioned to the autonomous trigger mode from a managed mode.

In the autonomous trigger mode, the radiation detector can constantly monitor and detect incident radiation, such as through an imaging array of the radiation detector. In one or all examples, the radiation detector can intermittently monitor and detect incident radiation. Upon detecting incident radiation, the radiation detector can begin an image acquisition process. The image acquisition process can begin when the radiation detector detects that incident radiation exceeds a threshold value. The image acquisition process can then stop after cessation of the incident radiation or after a predetermined time period. The image acquisition process can stop when the radiation detector detects that incident radiation falls below a threshold value. While the radiation detector is in the autonomous trigger mode, any images acquired by the radiation detector can be stored in a memory of the radiation detector.

When the radiation detector is connected to the dock (e.g., before being transitioned to the armed or autonomous trigger mode), the radiation detector can be in a connected, managed, or subordinate mode. In the connected mode, the radiation detector can perform image acquisition in response to commands received through the connector. Once an image is acquired, the radiation detector can transfer the image through the connector. The radiation detector can switch between the connected mode and the autonomous trigger mode based on the status of the electrical connection between the radiation detector and the dock, such as through the connector. In one or all examples, the radiation detector can perform image acquisition in the connected mode in response to detected incident radiation, similar to the autonomous trigger mode, and can then transfer acquired images through the connector.

In block 308, the radiation detector is connected to the dock. Block 302 can detect that the radiation detector has been connected to the dock. In response to the radiation detector being connected to the dock in block 306, images stored in the radiation detector are transferred in block 310. The images can be transferred through the connector to the dock, and from the dock to a computing device connected to the dock. Any images stored in the memory of the radiation detector can be automatically transferred or downloaded in response to the radiation detector being connected to the dock.

In one or all examples, connecting the radiation detector to the dock can prompt a request to a user of the computing device to accept or approve the transfer of the images from the radiation detector. The computing device can also prompt the user to specify whether all of the images stored in the memory relate to a single subject or object, to specify which subject or object each image relates to, or the like. In other words, the user can assign subject or object identities to each of the images transferred to the computing device. This can allow the radiation detector to be used to acquire radiation images from scans of multiple subjects or objects without having to connect the radiation detector to the dock between scans of different subjects or objects. In one or all examples, in response to connecting the radiation detector to the dock, the computing device can query the radiation detector to determine whether the memory includes images, and the images can be transferred in response to this query. In other words, handshaking can be performed between the radiation detector and the computing device before the images are transferred, and this handshaking can include approval by a user.

In one or all examples, the radiation detector can determine a location of the radiation detector upon connecting the radiation detector with the dock. For example, the dock can be positioned in a fixed or relatively fixed position, such as in a wall stand, in or on a table, in a mobile cart, or the like. Based on the identity and location of the dock to which the radiation detector is connected, the radiation detector can determine the location of the radiation.

By placing the radiation detector in the autonomous trigger mode, the radiation detector can be used for image acquisition while not being connected to the dock or otherwise connected to other external components or devices. In one or all examples, the radiation detector can perform radiation imaging without being physically or electrically connected (e.g., through a cable, connector, tether, or the like) or wirelessly connected (e.g., through Wi-Fi, Bluetooth, radio, infrared communication, or the like) to an external device or component. The radiation detector can be free from wireless communication components, which can reduce the cost of the radiation detector, communication certificate requirements, and the size of the radiation detector. The radiation detector can be placed in any desired positions to perform radiation imaging, and then the radiation detector can be connected to a dock to transfer images from the radiation detector. Although the radiation detector can be free from wireless communication components, the dock or a computing device connected to the dock can include wireless communication components or devices and the images acquired by the radiation detector can be communicated wirelessly through these components.

While the radiation detector is disconnected from the dock, the radiation detector can be powered by a supercapacitor. The supercapacitor can be used to quickly power components of the radiation detector, such as in response to the radiation detector detecting incident radiation. The supercapacitor can be charged quickly when the radiation detector is connected to a dock. The supercapacitor can support a charge sufficient to perform a large number of image acquisitions, such as hundreds of image acquisitions. In one or all examples, the radiation detector can be free from batteries, which can allow for easier and cheaper transportation and sale of the radiation detector. In one or all examples, the radiation detector can include a battery or other built-in power or energy storage device in place of or in addition to the supercapacitor.

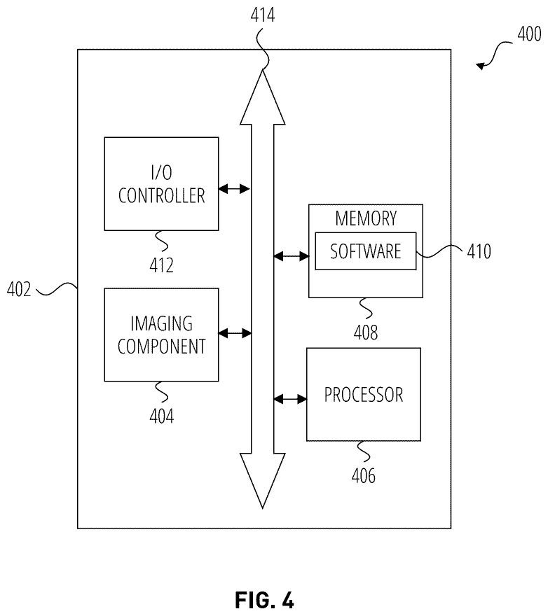

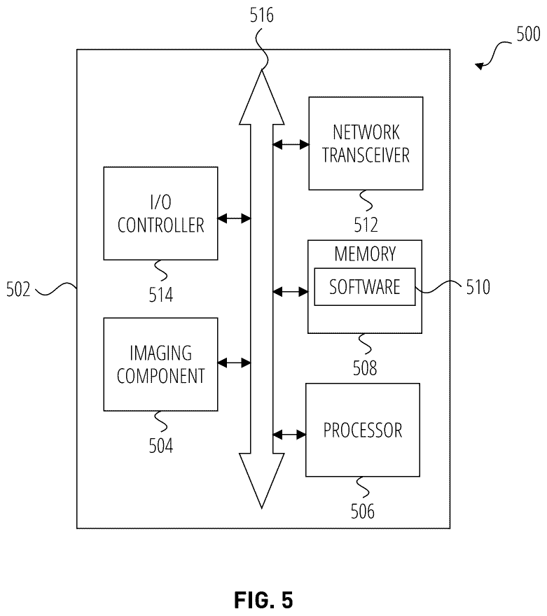

FIG. 4 illustrates a block diagram of a system 400 including a radiation detector 402 that supports methods for radiation imaging in accordance with aspects of the present disclosure. The system 400 can be contained within the radiation detector 102 in the imaging system 100, discussed above with respect to FIGS. 1 and 2, and can perform the method 300, discussed above with respect to FIG. 3, in addition to other or alternative methods and processes. In one or all examples, the radiation detector 402 can be a portable radiation detector, such as a direct or indirect flat panel detector. In one or all examples, the radiation detector 402 can be configured for bi-directional wireless communication with other systems or devices (e.g., an external device 502, such as a computing device, discussed below with respect to FIG. 5) using a dock (e.g., the dock 202, discussed above with respect to FIG. 2). The radiation detector 402 can include an imaging component 404, a processor 406, a memory 408, software 410, and an I/O controller 412. These components can be in electronic communication with one another via one or more buses (e.g., a bus 414).

The imaging component 404 can implement the functions of the radiation detector described with reference to the method 300 of FIG. 3, in addition to other or alternative methods and processes. The imaging component 404 can be implemented in hardware, software executed by the processor 406, firmware, or any combination thereof. For example, when the system 400 is disconnected from a dock (and an external component or device), the imaging component 404 can transition a mode of the radiation detector 402 from a connected mode to an automated trigger mode; when the system 400 is connected to the dock, the imaging component 404 can transition the mode of the radiation detector 402 from the automated trigger mode to the connected mode; when the system 400 is connected to the dock, the imaging component 404 can automatically transmit images stored on the system 400 through the dock or trigger a prompt on the external device for a user to accept the images from the system 400. Thus, the imaging component 404 can allow the system 400 to automatically switch between modes and transmit images in response to a connection status of the system 400. This can be used to simplify a process of acquiring radiation images with the radiation detector 402 when the system 400 is not connected to external devices and can allow the system 400 to function without wireless communication components or devices. The imaging component 404 can be implemented in hardware, software executed by the processor 406, firmware, or any combination thereof.

The processor 406 can include an intelligent hardware device (e.g., a general-purpose processor, a DSP, a central processing unit (CPU), a microcontroller, an ASIC, an FPGA, a programmable logic device, a discrete gate or transistor logic component, a discrete hardware component, or any combination thereof). The processor 406 can be configured to execute computer-readable instructions stored in a memory to perform various functions (e.g., functions or tasks supporting techniques for detecting incident radiation, acquiring and generating radiation images, storing acquired radiation images, detecting a connection status between the system 400 and an external device, switching a mode of the radiation detector 402, operating the radiation detector 402 in accordance with a current mode of the radiation detector 402, transferring images from the system 400 to an external device, and the like).

The memory 408 can include random access memory (RAM), read only memory (ROM), or the like. The memory 408 can be a volatile memory, a non-volatile memory, or can include both volatile and non-volatile memory. The memory 408 can store images or data acquired by an imaging array of the radiation detector 402 as the radiation detector 402 performs image acquisitions. The memory 408 can further store computer-readable, computer-executable software 510 including instructions that, when executed, cause the processor to perform various functions described herein. For example, the memory 408 can store software used to perform image acquisition through the imaging array, to adjust the mode of the radiation detector 402, to operate the radiation detector 402 based on the current operating mode of the radiation detector 402, to transfer images or data from the memory 408 to external components or devices, and the like. In one or all examples, the memory 408 can store a basic input/output system (BIOS), which can control interactions with peripheral components or devices.

The software 410 can include code to implement aspects of the present disclosure, including code to support techniques for detecting incident radiation, acquiring and generating radiation images, storing acquired radiation images, detecting a connection status between the system 400 and an external device, switching a mode of the radiation detector 402, operating the radiation detector 402 in accordance with a current mode of the radiation detector 402, transferring images from the system 400 to an external device, and the like. The software 410 can be stored in a non-transitory computer-readable medium such as system memory or other memory. The software 410 can be executable by the processor 406 to perform functions described herein.

The I/O controller 412 can manage input and output signals for the radiation detector 402. The I/O controller 412 can also manage peripherals not integrated into the radiation detector 402. In one or all examples, the I/O controller 412 can represent a physical connection or port to an external peripheral. For example, the I/O controller 412 can represent the connector 212, discussed above with respect to FIG. 2. In one or all examples, the I/O controller 412 can utilize an operating system such as iOS®, ANDROID®, MS-DOS®, MS-WINDOWS®, OS/2®, UNIX®, LINUX®, or another known operating system. In one or all examples, the I/O controller 412 can be implemented as part of a processor. In one or all examples, a user can interact with the radiation detector 402 via the I/O controller 412 or via hardware components controlled by the I/O controller 412.

FIG. 5 illustrates a block diagram of a system 500 including an external device 502 that supports methods for radiation imaging in accordance with aspects of the present disclosure. The system 500 can be used as a computing device or other external device in a radiation imaging system. The system 500 can be used to receive images from a radiation detector, display the images, perform operations on the images (e.g., assign a subject or object identity to the images), control components of a radiation imaging system (e.g., control a radiation source, alter settings in the radiation detector, or the like), and the like. In one or all examples, the system 400 can perform one or more operations in response to signals or commands from the system 500, such as transmitting images from the system 400 to the system 500 in response to a command from the system 500. The external device 502 can be an example of or include the components of a computer, a computing device, a computing system, a portable electronic device, a laptop computer, a tablet computer, a smartphone, a cellular phone, a wearable device, an internet-connected device, a server, a database, or another electronic device.

In one or all examples, the external device 502 can be configured for bi-directional wireless communication with other systems or devices. For example, the external device 502 can communicate with the radiation detector 402 through a dock, such as the dock 202, discussed above with respect to FIG. 2. The external device 502 can communicate with additional external devices through wireless communication (e.g., Wi-Fi, Bluetooth, radio, infrared communication, or the like).

The external device 502 can include an imaging component 504, a processor 506, a memory 508, software 510, a network transceiver 512, and an I/O controller 514. These components can be in electronic communication with one another via one or more buses (e.g., a bus 516). The external device 502 can communicate wirelessly with one or more other devices or computing systems over a network using the network transceiver 512.

The imaging component 504 can implement the functions of the external device described with reference to the method 300 of FIG. 3. For example, when the system 400 is connected to the system 500, the imaging component 504 can automatically accept images transmitted from the system 400. When the system 400 is connected to the system 500, the imaging component 504 can prompt a user of the system 500 whether to accept images from the system 400. The imaging component 504 can prompt a user of the system 500 whether all images stored in the system 400 belong to the same subject or object or different subjects or objects and can allow the user to assign subject or object identities to the images received from the system 400. The imaging component 504 can be implemented in hardware, software executed by the processor 506, firmware, or any combination thereof.

The processor 506 can include an intelligent hardware device (e.g., a general-purpose processor, a DSP, a central processing unit (CPU), a microcontroller, an ASIC, an FPGA, a programmable logic device, a discrete gate or transistor logic component, a discrete hardware component, or any combination thereof). The processor 506 can be configured to execute computer-readable instructions stored in a memory to perform various functions (e.g., functions or tasks supporting techniques for receiving radiation images, displaying radiation images, performing functions or operations on radiation images, assigning subject or object identities to radiation images, and the like).

The memory 508 can include random access memory (RAM), read only memory (ROM), or the like. The memory 508 can be a volatile memory, a non-volatile memory, or can include both volatile and non-volatile memory. The memory 508 can store images or data acquired from the system 400, such as images received from the radiation detector 402 in response to radiation imaging performed by the radiation detector 402. The memory 508 can further store computer-readable, computer-executable software 510 including instructions that, when executed, cause the processor to perform various functions described herein. In one or all examples, the memory 508 can store a basic input/output system (BIOS), which can control interactions with peripheral components or devices.

The software 510 can include code to implement aspects of the present disclosure, including code to support techniques for receiving radiation images, displaying radiation images, performing functions or operations on radiation images, assigning subject or object identities to radiation images, and the like. The software 510 can be stored in a non-transitory computer-readable medium such as system memory or other memory. The software 510 can be executable by the processor 506 to perform functions described herein or can cause a computer (e.g., when compiled and executed) to perform functions described herein.

The network transceiver 512 can communicate bi-directionally, via one or more antennas, wired, or wireless links. For example, the network transceiver 512 can represent a wireless transceiver and can communicate bi-directionally with another wireless transceiver. The network transceiver 512 can also include a modem to modulate the packets and provide the modulated packets to the antennas for transmission, and to demodulate packets received from the antennas. The system 400 can be connected to the system 500, and the system 500 can use the network transceiver 512 to wirelessly transmit images received from the system 400 to additional external devices.

The I/O controller 514 can manage input and output signals for the external device 502. The I/O controller 514 can also manage peripherals not integrated into the external device 502. In one or all examples, the I/O controller 514 can represent a physical connection or port to an external peripheral. For example, the I/O controller 514 can represent the connector 214, discussed above in reference to FIG. 2. In one or all examples, the I/O controller 514 can utilize an operating system such as iOS®, ANDROID®, MS-DOS®, MS-WINDOWS®, OS/2®, UNIX®, LINUX®, or another known operating system. In one or all examples, the I/O controller 514 can represent or interact with a modem, a keyboard, a mouse, a touchscreen, or a similar device. In one or all examples, the I/O controller 514 can be implemented as part of a processor. In one or all examples, a user can interact with the external device 502 via the I/O controller 514 or via hardware components controlled by the I/O controller 514.

The external device 502 can receive data (e.g., radiation images) from external devices, such as the radiation detector 102 discussed above with respect to FIGS. 1 and 2 or the radiation detector 402 discussed above with respect to FIG. 4. The imaging component 504 can display the radiation images, perform functions or operations on the radiation images, assign subject or object identities to radiation images, and the like. Connecting the system 400 to the system 500 can prompt a user of the radiation detector 402 to accept images from the system 400, perform functions on the images transmitted from the system 400 to the system 500 (e.g., assign subject or object identities to the images), and the like.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not intended to be exhaustive or to limit the embodiments to the precise forms disclosed. It will be apparent to one of ordinary skill in the art that many modifications and variations are possible in view of the above teachings.

What is claimed is:

1. A non-transitory computer-readable medium storing code for radiation imaging, the code comprising instructions executable by a processor to:
detect a connection status of an electrical connection between a radiation detector and a dock;
enter an autonomous trigger mode in response to detection of the electrical connection being disconnected; and
automatically acquire images through the radiation detector while the radiation detector is in the autonomous trigger mode.

2. The non-transitory computer-readable medium of claim 1, wherein the code is configured to cause the radiation detector to begin an image acquisition upon detecting incident radiation and stop the image acquisition upon detecting cessation of the incident radiation.

3. The non-transitory computer-readable medium of claim 1, wherein the code further comprises instructions executable by the processor to store the images a memory in the radiation detector.

4. The non-transitory computer-readable medium of claim 1, wherein the code further comprises instructions executable by the processor to download the images acquired by the radiation detector to a computing device through the dock in response to detection of the electrical connection being connected.

5. The non-transitory computer-readable medium of claim 1, wherein the code further comprises instructions executable by the processor to transition from the autonomous trigger mode to a connected mode in response to detection of the electrical connection being connected.

6. The non-transitory computer-readable medium of claim 1, wherein:
the images are acquired by an imaging array of the radiation detector; and
the code further comprises instructions executable by the processor to power the imaging array by a supercapacitor of the radiation detector.

7. A method of radiation imaging, the method comprising:
detecting a status of an electrical connection between a radiation detector and a dock; and
transferring images from the radiation detector through the electrical connection upon detecting the radiation detector has been electrically connected to the dock.

8. The method of claim 7, further comprising:
automatically transitioning an image acquisition mode of the radiation detector upon detecting the radiation detector has been electrically disconnected from the dock.

9. The method of claim 8, wherein when the radiation detector is electrically disconnected from the dock, the radiation detector is configured to automatically:
begin an image acquisition upon detecting incident radiation; and
stop the image acquisition upon detecting cessation of the incident radiation.

10. The method of claim 7, further comprising:
identifying the dock with the radiation detector; and
determining a location of the detector based on the identification.

11. The method of claim 7, further comprising requesting a user action upon detecting the radiation detector has been electrically connected to the dock.

12. The method of claim 7, further comprising assigning one or more subject identities to the images transferred through the electrical connection.

13. A radiation detector comprising:
a housing;
an imaging array within the housing;
a connector in the housing, the connector comprising at least 26 pins;
a memory; and
a processor coupled to the imaging array, the connector, and the memory, wherein the processor is configured to:
adjust a mode of the radiation detector based on a connection status of the connector;
acquire images from the imaging array based on the mode; and
store the images to the memory.

14. The radiation detector of claim 13, wherein the processor is further configured to transmit the images from the memory to an external computing device based on the connection status of the connector.

15. The radiation detector of claim 13, wherein the processor is configured to transfer the images stored in the memory through the connector.

16. The radiation detector of claim 13, wherein the housing is free of a wireless communication device.

17. The radiation detector of claim 13, wherein the processor is further configured to acquire the images by initiating an image acquisition upon the imaging array detecting incident radiation and stopping the image acquisition upon the imaging array detecting cessation of the incident radiation.

18. The radiation detector of claim 13, further comprising a supercapacitor in the housing, the supercapacitor configured to power the imaging array, power the processor, and receive power through the connector.

19. The radiation detector of claim 13, further comprising a battery in the housing, the battery configured to power the imaging array, power the processor, and receive power through the connector.

20. The radiation detector of claim 13, wherein the connector comprises at least 26 pins.
